(19) United States
(12) Patent Application Publication
(10) Pub. No.: US 2010/0267075 A1
(43) Pub. Date: Oct. 21, 2010

(54) MICROFLUIDIC LYSIS

(75) Inventors: Palaniappan Sethu, Louisville, KY (US); William N. White, Louisville, KY (US)

Correspondence Address: VIKSNINS HARRIS & PADYS PLLP, P.O. BOX 111098, ST. PAUL, MN, 55111-1098, US

(73) Assignee: UNIVERSITY OF LOUISVILLE RESEARCH FOUNDATION, INC., Louisville, KY (US)

(21) Appl. No.: 12/680,100

(22) PCT Filed: Sep. 19, 2008
(86) PCT No.: PCT/US2008/077004

§ 371 Date: Jun. 28, 2010

Related U.S. Application Data
(60) Provisional application No. 60/975,397, filed on Sep. 26, 2007.

(51) Int. Cl. C12Q 1/02; C12N 5/071; C12N 5/09

(52) U.S. Cl. 435/29; 435/325; 435/366

(57) ABSTRACT

The invention provides methods for isolating and identifying nucleated cells from biological samples using a microfluidic device.

### RELATED APPLICATION(S)

This patent document claims the benefit of priority of U.S. application Ser. No. 60/975,397, filed Sep. 26, 2007, which application is herein incorporated by reference.

### BACKGROUND

The circulatory and nervous systems are the primary mechanisms for homeostasis in the body. Good health and disease can be correlated with presence or absence of circulating nucleated cells in blood. For example, AIDS diagnostics correlate to the ratio of CD4+ to CD8+ cells, cancer detection can be accomplished through identification of circulating tumor cells, and vascular diseases can be correlated to presence of endothelial cells in circulation.

Much like genomics and proteomics that look at gene and protein expression, cellomics, or characterization of cellular populations, can also be used for diagnostic and prognostic purposes. This involves techniques that remove contaminating erythrocytes from whole blood before enumeration of circulating nucleated cells can be accomplished. Thus, methods for removing erythrocytes from samples are needed.

### SUMMARY OF CERTAIN EMBODIMENTS OF THE INVENTION

As described herein, new protocols that accomplish depletion of contaminating erythrocytes, while maintaining circulating nucleated cells, have been developed. Using a microfluidic device with the new protocols, nucleated cells from human peripheral blood have been identified. Typical flow cytometry scatter plots accomplish separation into 3 major populations. Using the techniques described herein, it has been possible to separate the cells into 4 major populations. The 4^{th} major population contains cells that are CD31+ and CD61+. These cells potentially represent mature and progenitor endothelial cells, megakaryocytes, megakaryoblasts, hematopoietic and non-hematopoeitic stem/progenitor cells, fibrocytes, circulating tumor cells, kupfer cells, osteoclasts, osteoblasts and/or fibroblasts.

A further modified protocol has also been developed for isolation of circulating nucleated cells. Results demonstrate a significant increase in numbers of circulating cells when compared to existing protocols.

Accordingly, certain embodiments of the present invention provide methods of using a microfluidic device to isolate nucleated cells from a biological sample from a subject, comprising processing the sample using the microfluidic device so as to deplete erythrocytes from the sample while preserving the nucleated cells from the sample, wherein the sample is contacted with a deionized water solution comprising containing 2% paraformaldehyde.

Certain embodiments of the present invention provide methods of using a microfluidic device to isolate nucleated cells from a biological sample from a subject who has or is at risk for developing Sickle Cell Disease, comprising processing the sample using the microfluidic device so as to deplete erythrocytes from the sample while preserving the nucleated cells from the sample.

Certain embodiments of the present invention provide methods of using a microfluidic device to isolate and identify nucleated CD61+/CD31+ cells from a biological sample from a subject, comprising processing the sample using the microfluidic device so as to deplete erythrocytes from the sample while preserving the nucleated cells from the sample and identifying nucleated CD61+/CD31+ cells from the biological sample.

Certain embodiments of the present invention provide methods of using a microfluidic device to isolate and identify nucleated CD146+/CD61+ cells from a biological sample from a subject, comprising processing the sample using the microfluidic device so as to deplete erythrocytes from the sample while preserving the nucleated cells from the sample and identifying nucleated CD146+/CD61+ cells from the biological sample.

In certain embodiments, the sample is contacted with a deionized water solution containing 2% paraformaldehyde.

In certain embodiments, the depletion is accomplished using a method comprising contacting the sample with deionized water for about ten seconds.

In certain embodiments, the biological sample is whole blood.

In certain embodiments, the subject is a mammal. In certain embodiments, the subject is a human. In certain embodiments, the subject is a male. In certain embodiments, the subject is a female. In certain embodiments, the subject is a subject who has or is at risk for developing Sickle Cell Disease.

In certain embodiments, the nucleated cells comprise CD31+/CD61+ cells.

In certain embodiments, the nucleated cells comprise CD146+/CD61+ cells.

In certain embodiments, the nucleated cells comprise endothelial cells.

In certain embodiments, the nucleated cells comprise tumor cells.

In certain embodiments, the methods further comprise identifying the nucleated cells. In certain embodiments, the nucleated cells are identified using at least one antibody (e.g., at least one monoclonal and/or polyclonal antibody that specifically binds to a specific type of cell).

In certain embodiments, the methods further comprise diagnosing the subject.

### BRIEF DESCRIPTION OF THE FIGURES

FIG. 1 depicts a microfluidic lysis cassette. Device design and construction to facilitate the rapid lysis of erythrocytes and isolation of leukocytes following erythrocyte lysis. The bottom panel shows different locations in the cassette. (A) Inlets for addition of whole blood sample and deionized water. (B) The double herringbone structures in the microchannel floor mediate chaotic mixing and uniform cell distribution. (C) Outlets, e.g., for addition of 2×PBS to restore isotonic conditions and to collect erythrocyte depleted leukocyte samples. The samples and reagents can be automatically metered out in precise predetermined volumes, and the constant flow conditions prevent sedimentation or cell loss in the micro-channels. Complete and rapid mixing of the cells with the lysis buffer streams were visually confirmed by microscopy. Whole blood can be exposed to deionized water, e.g., for about 10 seconds, and brought back into isotonic conditions, e.g., with 2×PBS.

FIG. 2 depicts a view of the microfluidic device.

FIG. 3 depicts the identification of a 4^{th} major population of cells on the flow cytometry scatter plots. The majority of cells in the 4^{th} region phenotyped as being CD31+/CD61+.

FIG. 4 depicts results indicating that the majority of cells in the 4^{th} region phenotyped are CD31+/CD61+ cells.

FIG. 5 depicts the ability to detect the presence of significantly larger number of CD146+/CD61+ endothelial cells than reported in literature.

FIG. 6 depicts a comparison of controls and sickle cell patients and shows an increased number of CD31+ and CD61+ cells.

FIG. 7. (A) Inlets for addition of whole blood sample and deionized water. (B) The double herringbone structures in the microchannel mediate chaotic mixing and uniform cell distribution. (C) Outlets, e.g., for addition of 2×PBS to restore isotonic conditions and to collect erythrocyte depleted leukocyte samples. (D) Live photograph of inlet with mixture of whole blood and deionized water. (E) Live photograph of isolated leukocytes at outlet.

FIG. 8 depicts the operational setup.

FIG. 9. Microfluidics lysis effect on total and differential leukocyte and endothelial cell recovery. No apparent loss of any cell subpopulation following erythrocyte lysis is seen from microfluidic samples. Number of endothelial cells (CD61/CD31) isolated were an order of magnitude higher with microfluidic erythrocyte lysis (note different scales on graphs). The results encompass six different control blood samples and SCD samples.

FIG. 10. Flow Cytometry Scatter Plots for microfluidics. Top plot depicts forward scattered light (FSC) versus side scattered light (SSC). The R1, R2, R3, and R4 regions represent CD61⁺/CD31⁺ cells, lymphocytes, monocytes, and granulocytes, respectively. Bottom plots show FSC versus CD61/CD31 (FITC), exhibiting that the endothelial cell progenitor phenotype saved by microfluidics is both CD61 and CD31 positive.

FIG. 11. Chart depicting cell population counts for control versus patient sample. Initial results from SCD patients found more CD61⁺/CD31⁺ cells in peripheral blood compared to controls. Abundance of CD61⁺/CD31⁺ cells possibly indicate tissue repair following endothelial cell damage by sickle cells.

### DETAILED DESCRIPTION

Certain embodiments of the invention provide methods of isolating, characterizing and/or identifying nucleated cells in biological samples, e.g., blood. Certain embodiments of the invention provide identification of rare cells in blood, e.g., circulating tumor cells. Certain embodiments of the invention provide cellular diagnostic and prognostic markers for disease and health, e.g., by utilizing information from the characterization and identification of cells.

Target applications include circulating nucleated cell phenotyping and characterization in disease and health (cellomics), detection of rare cells including circulating tumor cells in cancer, identification of circulating endothelial cells in vascular disorders, and identification of stem/progenitor cells in injury, trauma and tissue repair.

Using a microfluidic lysis device (see, e.g., Sethu et al., Anal. Chem., 78, 5453-5461 (2006)), new protocols have been developed. In certain embodiments, the protocols include the use of 2% paraformaldehyde with DI water, rather than with 2×PBS. Certain embodiments of the invention provide for the isolation of circulating nucleated cells, e.g., from peripheral blood of humans, that have not been reliably isolated using conventional protocols. Certain embodiments of the invention provide for the identification of cells in numbers not possible using other techniques, including total leukocytes, hematopoietic stem cells (CD34+), mature and progenitor endothelial cells (CD146+/CD36+) and (CD34+/CD133+), fibrocytes (CD11b+/HLA-DR+), megakaryocytes (CD61+), PECAM+cells, and/or CD66b+/CD49d+ cells. Table 1 shows the distribution of various nucleated cell populations from whole blood.

TABLE 1

| | | Events | Microfluid- | Conventional |
|---|---|---|---|---|
| | Phenotype | (per | ics | Techniques |
| Cell Type | Marker | 10000) | (cells/mL) | (cells/mL) |
| Granulocytes | CD66+ | 4145 | 1533650 | |
| Granulocyte | CD66+/CD49d+ | 238 | 88060 | |
| sub-population | | | | |
| Monocytes/ | CD14+ | 412 | 152440 | |
| Macrophages | | | | |
| N/A | HLA-DR+ | 170 | 62900 | |
| Fibrocytes | HLA-DR+/ | 155 | 57350 | |
| | CD11b+ | | | |
| T-cells | CD4+/CD3+ | 689 | 254930 | |
| | CD4−/CD3+ | 940 | 347800 | |
| T-regulatory | CD4+/CD25+ | 117 | 43290 | |
| cells | | | | |
| Natural Killer | CD56+ | 375 | 138750 | |
| cells | | | | |
| Hematopoetic | CD34+ | 2175 | 804750 | |
| Stem cells | | | | |
| Hemangioblast | CD133+/CD34+ | 22 | 8140 | |
| Mature | CD146+/CD36+ | 166 | 61420 | |
| Endothelial cell | | | | |
| | CD146+/CD31+ | 11 | 4070 | |
| | CD146+/CD106+ | 107 | 39590 | |
| | CD146+/CD54+ | 11 | 4070 | |
| | CD146+/CD62p+ | 97 | 35890 | |
| Megakaryocyte | CD61+ | 3193 | 1181410 | |
| PECAM+ | CD31+/CD61+ | 3131 | 1158470 | |
| Total | | 10000 | 3700000 | 5000000 [1] |
| Leukocytes | | | | |

[1] see www.drhull.com/EncyMaster/C/CBC.html

Certain embodiments of the invention provide for the identification of a 4^{th} major population in flow cytometry scatter plots, whereas conventional protocols show only 3 major populations. Certain embodiments of the invention provide for the identification of cells that include, but are not limited to, CD31+/CD61+ cell populations that may potentially represent megakaryocytes, megakaryoblasts, non-hematopoetic stem cells, circulating tumor cells, kupfer cells, osteoclasts, osteoblasts and/or fibroblasts.

Certain embodiments of the invention provide for the isolation of a significantly larger number of circulating nucleated cells from blood than possible with conventional protocols.

The invention will now be illustrated by the following non-limiting Examples.

### Example 1

### Description of the Device and Operation

Microfluidic Cassette Fabrication: The microfluidics device was fabricated using soft lithographic techniques. A silicon wafer was treated with oxygen plasma in an asher (March Instruments, Concord, Mass.) and spin coated with the negative photoresist SU-8 (MicroChem, Newton, Mass.). AutoCAD (Autodesk, Inc., San Rafael, Calif.) was used to generate a transparency mask (CAD ART Services Inc., Poway, Calif.) for photolithography, to create negative replicas of the channels. The elastomer poly(dimethylsiloxane) (PDMS; Dow Corning, Midland, Mich.) was mixed 10:1 with a cross-linker, poured on top the silicon wafer, and cured at 60° C. for 12 h. The elastomer with the replicated channels was released, and channel access holes were punched with a 22-gauge needle. The PDMS wafer was irreversibly bonded to a glass slide via oxygen plasma. Access tubing (Tygon; Miami Lakes, Fl) of slightly larger diameter was press-fitted into the holes.

### Microfluidic Cassette Design and Operation:

FIG. 1 shows that the microfluidics cassette has three inlets and one outlet. The sample collection end has a sample outlet and an inlet that can be used, e.g., for 2× phosphate-buffered saline (PBS) addition and/or for 2% paraformaldehyde with DI water. The sample loading end has two inlets, for whole blood and for deionized water. Syringe pumps drive liquid flow, with blood at 20 μL/min, and deionized water and 2×PBS at 600 μL/min Experiments were performed using a similar setup as described by Sethu et al. (Anal. Chem., 76, 6247-6253 (2004)). The water was divided into two streams that flank the whole blood stream leading into the serpentine lysis channel. At the flow rates used, the cells were in contact with deionized water for 10 s. Despite the limited time required for ionic diffusion in the microscale, the high concentration of blood cells may still produce nonuniform conditions, especially in the upstream microchannel. To rapidly and uniformly mix the cells, the channel floors are patterned with double herringbone microridges, which generate nonuniform resistance that affects fluid rotation. Also, the variable ridge length and their arrangement produce immediate chaotic mixing for even cell distribution. The channels are 160 cm long with cross section of 500×200 μm. Ridges are 25 μm high and 20 μm wide. Internal volume is 68.89 μL.

### Leukocyte Isolation and Recovery:

Two 0.6-mL aliquots from unstimulated and stimulated blood samples were enriched for leukocytes via microfluidic lysis or via the widely used FACSlyse protocol, which lyses erythrocytes using hypertonic conditions in macroscale for 5 min and fixes the remaining leukocytes for flow cytometry. The device processes 20 μL of blood/min, so 0.6 mL requires 30 min. Note that each blood cell is exposed to the hypotonic lysis conditions in the cassette device for just 8-10 s. The procedure requires no user assistance, is fully automated, and can be run in parallel.

FIG. 1 shows whole blood and deionized water are simultaneously introduced into the cassette via their respective inlets, to achieve a 1:30 blood-to-deionized water ratio, which was determined to produce complete erythrocyte lysis within 10 s. Based on channel dimensions; a 600 μt/min flow rate gives a 10-12-s cell residence time. At the cell collection end, 2×PBS, with or without 2% paraformaldehyde (Fisher Scientific Corp., Pittsburgh, Pa.), quickly returns the cells to isotonic conditions, for flow cytometry and RNA isolation, respectively. Lysed samples, enriched for leukocytes, are collected from the outlet in 0.5-mL Eppendorf tubes, with cell debris removed in the supernatant by low-speed centrifugation. The leukocyte pellets are washed and dispersed in Ix PBS for subsequent analyses.

Certain embodiments of the invention include the method as described below, and other embodiments include combinations of the following method steps detailed below. In certain embodiments, the methods of the invention involve the use of 2% paraformaldehyde with DI water, rather than 2×PBS (see, e.g., steps 9 and 13A). While the methods can be performed in the device as described, the methods can also be performed in other devices, e.g., other microfluidic devixes, which devices are well known to the art worker.

### Exemplary Protocol

### Sample Collection:

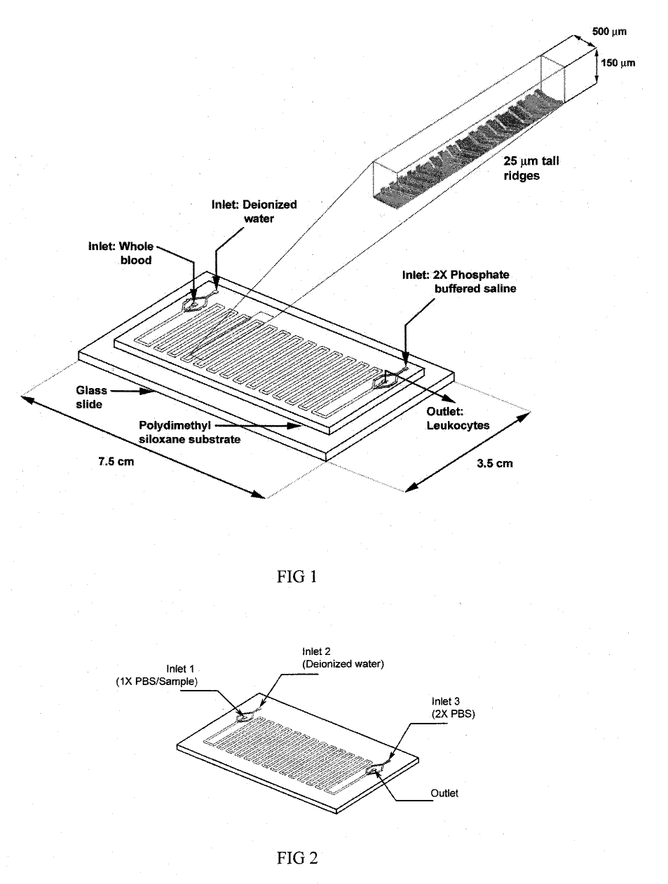

1. Collect 4 milliliters of blood sample from median cubital vein, on the anterior forearm vein of patient with heparin as anticoagulant in two 2 milliliter green top vaccutainers.
2. Discard first 2 milliliter tube as it contains dislodged endothelial cells, which can lead to false positives.
3. Resuspend the 2^{nd} tube to mix heparin with blood and save immediately on ice.
4. Process the blood sample within one hour of collection.
   Prime the Microfluidics Cassette with PBS: (See FIG. 2)
5. Fill a 1 ml syringe with 1×PBS and remove bubbles. Connect the PBS-loaded 1 ml syringe to inlet 1 on the microfluidics device. Push the syringe containing the 1×PBS gently by hand until the solution flows out of inlet 2, and then immediately clamp inlet 2 with a standard office binder clip (Sparco model 87002)
6. Continue pushing the 1×PBS until fluid reaches the outlet port of the microfluidics device. Once the solution flows out of the outlet, clamp the outlet tubing with another binder clip.
7. Continue pushing the 1 ml syringe until the 1×PBS flows out of inlet 3, and clamp the inlet 3 port with another office binder clip. The microfluidics cassette is now fully primed.

### Preparation of the Syringe Pumps and Solutions:

8. To calibrate the pumps follow these steps:
A) For the large Harvard syringe pump (Harvard, PHD 22/2000 Part #702001): Set the diameter configuration to 22.5 mm, flow rate 620 μl/min.
B) For the small Harvard Syringe pump (Harvard, PHD 22/2000 Part #702209): Set the diameter configuration to 4.61 mm, Flow rate 25 μl/min. It is strongly recommended that the pumps be calibrated prior to use by collecting a timed volume of the fluid on an electronic balance to verify the flow rates. Flow should be precise within 5% of anticipated rates.
9. Fill one 30 ml syringe (Becton-Dickinson, Part #EF23525E) with sterile deionized water containing 2% paraformaldehyde (PFA). This can be accomplished by withdrawing the solution directly from the 50 ml conical tube. (Label the syringe)
10. Fill a second 30 ml syringe with 2× phosphate-buffered-saline (PBS), using the procedure outlined in step 8. (Label the syringe)
11. Fill a 1 ml syringe (Becton-Dickinson, Part #EF2379A) with 1×PBS from the aliquots stored in the 4 ml Falcon tubes, using the procedure outlined in step 8.
12. Remove the clamps and connect the 30 ml deionized water syringe to inlet 2 and the 30 ml 2×PBS syringe to inlet 3 (avoid trapping bubbles in the tubing or microfluidics cassette). Remove the clamp from the outlet tubing. Connect the 1×PBS 1 ml syringe into inlet 1.

### Erythrocyte Lysis:

13. Set all of the syringes on the proper Harvard pumps as outlined below:
   A) Set the two 30 ml syringes (one containing sterile, de-ionized water, 2% PFA and the other with 2×PBS) together on the large Harvard syringe pump.
   B) Set the 1 ml syringe (containing 1×PBS) on the small Harvard syringe pump.
   C) Place the outlet tubing in a waste collector (50 ml conical tube that is labeled “waste”).
14. Turn on the large Harvard syringe pump (with the 30 ml syringes) and let it run for one minute. (Make sure there is no leakage in the device or in the tubing).
15. Turn on the small Harvard syringe pump and let it run for an additional minute. (Make sure there is no leakage in the device or in the tubing). Stop the pumps. The microfluidics device is now ready to be used.
16. Remove the 1 ml syringe containing 1×PBS from the small Harvard syringe pump.
17. After obtaining a blood sample, fill the 1 ml syringe (Becton-Dickinson, Part #EF2379A) with 0.05 ml of sterile 1×PBS without trapping any bubbles. Next, fill the syringe with 0.5 mls of blood obtained from the Eppendorf tube. (The 1 ml syringe will now contain a final volume of 0.55 mls of the blood and 1×PBS buffer.) Keep the syringe vertical while filling to avoid mixing of the 0.05 ml PBS with the blood.
18. Connect the syringe containing the blood to inlet 1 and mount the syringe carefully (avoid pushing the blood through the tubing into the device) on the small Harvard syringe pump (the syringe is mounted vertical into the Pump).
19. Remove the outlet tubing from the “waste” tube and put a clean 50 ml Corning centrifuge tube in its place and set it on a bucket of ice.
20. Switch on the large Harvard syringe pump first and let it run for 1 minute. Start collecting the sample from the outlet into the 50 ml Corning centrifuge tube (Corning Labs. Cat#430828).
21. Switch on the small Harvard syringe pump.
22. Once the blood sample in the 1 ml syringe has completely traveled through the device, stop both pumps. This should take approximately 20 minutes.
23. Centrifuge the collected sample for 5 minutes at 350×g at room temperature with the brake off.
24. Remove supernatant by placing pipette tip at the opposite side of the white pellet. Remove as much supernatant as possible, especially red cell debris, without disturbing the white pellet.
25. Resuspend the sample in 1 mL of flow buffer.
26. For sample analysis using flow cytometry, add 100 microliters of sample to a flow cytometry tube.
27. To the 100 microliters of sample add specified antibody.
28. Allow sample to incubate for 30 minutes at 4° C. Wash twice with flow buffer prior to flow cytometry (add 250 ul of flow buffer, vortex, spin at 350 g for 5 mins, resuspend in 250 ul of flow buffer.)

### Example 2

The new methods described herein have been used to isolate circulating nucleated cells from both controls (healthy volunteers) and patients with Sickle Cell Disease.

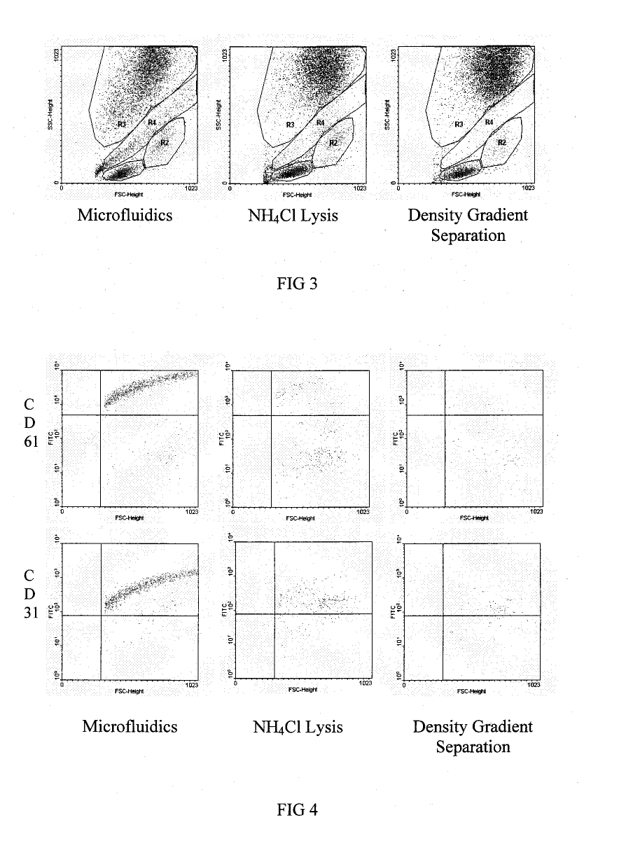

FIG. 3 depicts the identification of a 4^{th} major population of cells on the flow cytometry scatter plots. As depicted in FIG. 4, the majority of cells in the 4^{th} region phenotyped as being CD31+/CD61+.

FIG. 5 depicts the ability to detect the presence of significantly larger number of CD146+/CD61+ endothelial cells than reported in literature.

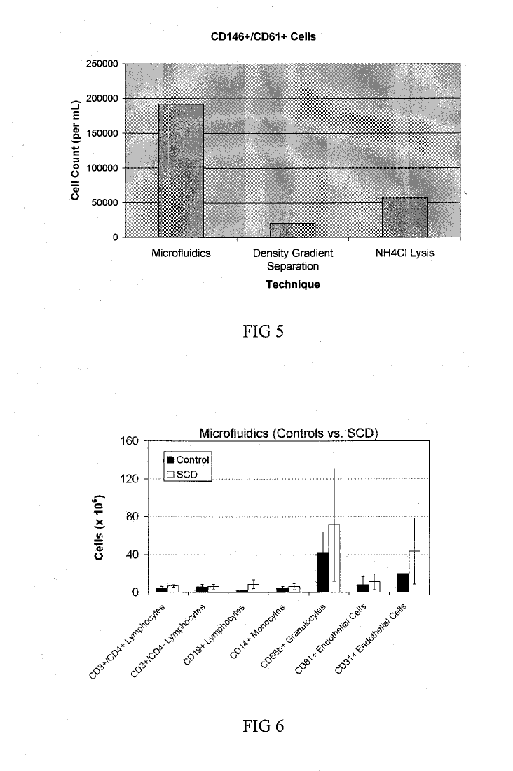

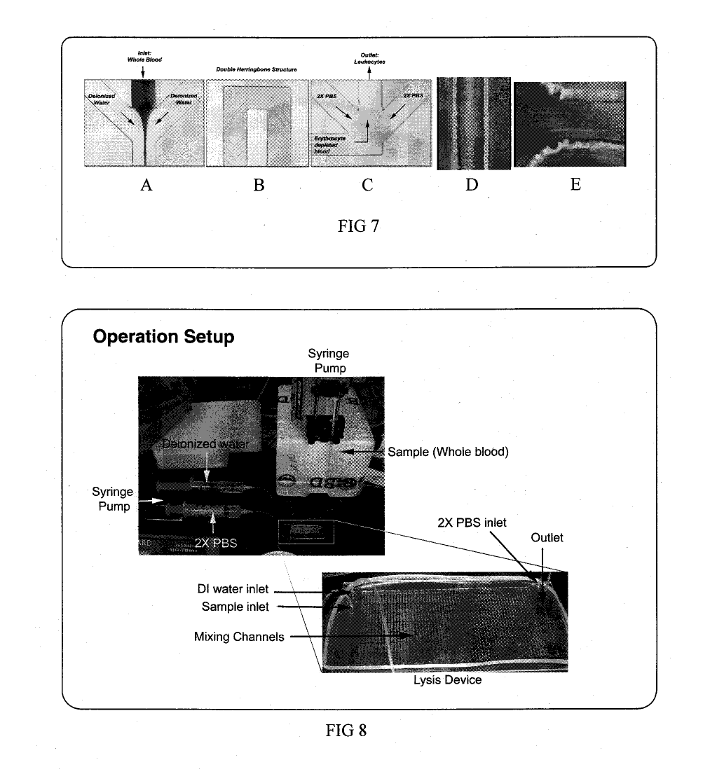

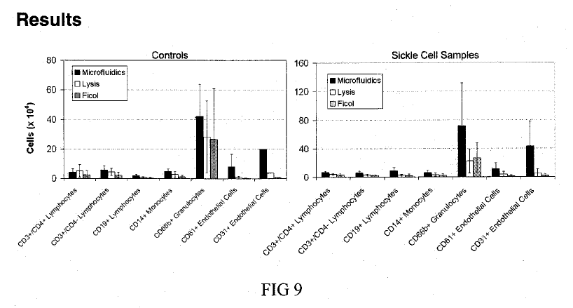

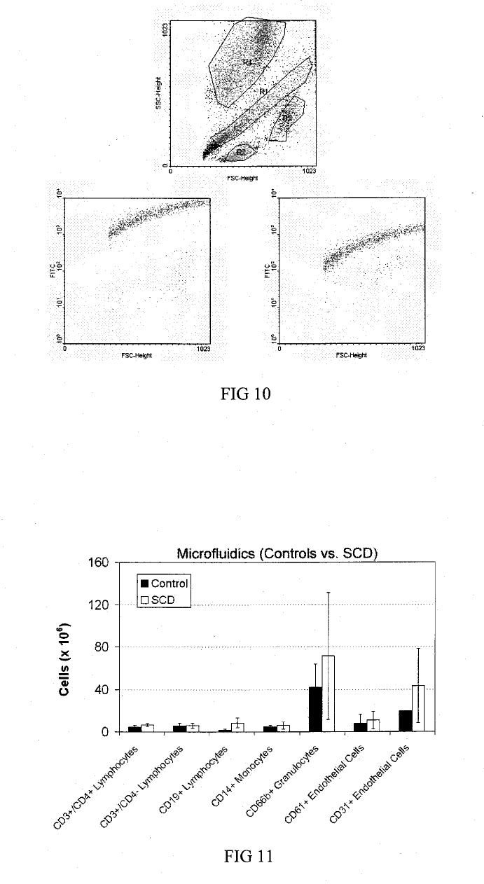

FIG. 6 depicts a comparison of controls and sickle cell patients and shows an increased number of CD31+ and CD61+ cells.

### Example 3

### Isolation of Endothelial Cells from Peripheral Blood using Microfluidics for Sickle Cell Disease (SCD)

Endothelial cells are known to play key roles in the pathogenesis of several vascular diseases, such as Sickle Cell Disease (SCD). Endothelial cells in peripheral blood may indicate a role in the initiation of vaso-occlusion in SCD patients, and it is important to determine the presence of these cells. The small numbers of circulating endothelial cells in whole blood requires techniques that can accomplish reliable isolation. Commonly used methods for depletion of erythrocytes in blood and isolation of nucleated cell populations include density gradient separation and NH4Cl lysis. In comparison to these methods, microfluidics preserves a larger quantity of cells and sub-populations. Endothelial cells, in particular, are very sensitive to stress, and a majority are lost in current clinical isolation protocols. Preliminary results from controls show microfluidics recovered 12.1×107 CD61+/CD31+ cells/ml from whole blood, as opposed to 9.75×104/ml and 1.25×106/ml CD61+/CD31+ cells from density gradient separation and NH4Cl lysis, respectively. These CD61+/CD31+ cells indicate an endothelial cell phenotype, both mature and progenitor. Utilizing microfluidics and the methods described herein, new cell populations lost in conventional techniques can be identified and used to characterize and diagnose vascular diseases.

The advantages of microfabrication include the ability to expose cells in blood to deionized water at the single cell level for a minimum required time necessary for lysis of erythrocytes (e.g., 10 seconds), returning cells from a hypotonic environment to a isotonic environment within milliseconds, and minimal cell damage and activation in short exposure to deionized water.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of using a microfluidic device to isolate nucleated cells from a biological sample from a subject, comprising processing the sample using the microfluidic device so as to deplete erythrocytes from the sample while preserving the nucleated cells from the sample, wherein the sample is contacted with a deionized water solution comprising 2% paraformaldehyde.

2. A method of using a microfluidic device to isolate nucleated cells from a biological sample from a subject who has or is at risk for developing Sickle Cell Disease, comprising processing the sample using the microfluidic device so as to deplete erythrocytes from the sample while preserving the nucleated cells from the sample.

3. A method of using a microfluidic device to isolate and identify nucleated CD61+/CD31+ cells from a biological sample from a subject, comprising processing the sample using the microfluidic device so as to deplete erythrocytes from the sample while preserving the nucleated cells from the sample and identifying nucleated CD61+/CD31+ cells from the biological sample.

4. A method of using a microfluidic device to isolate and identify nucleated CD146+/CD61+ cells from a biological sample from a subject, comprising processing the sample using the microfluidic device so as to deplete erythrocytes from the sample while preserving the nucleated cells from the sample and identifying nucleated CD146+/CD61+ cells from the biological sample.

5. The method of claim 2, wherein the sample is contacted with a deionized water solution comprising 2% paraformaldehyde.

6. The method of claim 1, wherein the depletion is accomplished using a method comprising contacting the sample with deionized water for about ten seconds.

7. The method of claim 1, wherein the biological sample is whole blood.

8. The method of claim 1, wherein the subject is a mammal.

9. The method of claim 8, wherein the subject is a human.

10. The method of claim 9, wherein the subject is a male.

11. The method of claim 9, wherein the subject is a female.

12. The method of claim 1, wherein the subject is a subject who has or is at risk for developing Sickle Cell Disease.

13. The method of claim 1, wherein the nucleated cells comprise CD31+/CD61+ cells.

14. The method of claim 1, wherein the nucleated cells comprise CD146+/CD61+ cells.

15. The method of claim 1, wherein the nucleated cells comprise endothelial cells.

16. The method of claim 1, wherein the nucleated cells comprise tumor cells.

17. The method of claim 1, further comprising identifying the nucleated cells.

18. The method of claim 1, wherein the nucleated cells are identified using at least one antibody.

19. The method of claim 1, further comprising diagnosing the subject.
